(19) United States
(12) Patent Application Publication
(10) Pub. No.: US 2026/0188512 A1
(43) Pub. Date: Jul. 2, 2026

(54) INFORMATION PROCESSING DEVICE, SERVER DEVICE, NON-TRANSITORY COMPUTER-READABLE MEDIUM, AND METHOD OF ASSISTING ASSESSMENT OF PATIENT'S STATUS

(71) Applicant: NIHON KOHDEN CORPORATION, Tokyo (JP)

(72) Inventors: Daisuke HORIGUCHI, Saitama (JP); Syuhei KAWAMURA, Saitama (JP); Mitsuhiro OURA, Saitama (JP); Wataru MATSUZAWA, Saitama (JP); Yuichiro SAWANO, Saitama (JP)

(73) Assignee: NIHON KOHDEN CORPORATION, Tokyo (JP)

(21) Appl. No.: 19/423,315

(22) Filed: Dec. 17, 2025

(30) Foreign Application Priority Data
Dec. 27, 2024 (JP) 2024-232457

(51) Int. Cl. G16H 50/30; A61B 5/00; G06N 5/045

(52) U.S. Cl. CPC G16H 50/30; A61B 5/7267; A61B 5/742; A61B 5/7475; G06N 5/045

(57) ABSTRACT

An interface receives physiological information of a patient. A processor acquires contribution information corresponding to contributions of N first parameters (N is an integer of at least 2) with respect to a status of the patient based on the physiological information. The processor causes a visualizing device to visualize the contribution information under a condition that M first parameters (M is an integer of at least 2 and at most N) included in the N first parameters are substituted by L second parameter (L is an integer less than M).

### CROSS-REFERENCE TO RELATED APPLICATION

The present application is based on Japanese Patent Application No. 2024-232457 filed on Dec. 27, 2024, the entire contents of which are incorporated herein by reference.

### BACKGROUND

The presently disclosed subject matter relates to an information processing device configured to process physiological information of a patient. The presently disclosed subject matter also relates to a server device capable of communicating with a client device and provided with the information processing device. The presently disclosed subject matter also relates to a non-transitory computer-readable medium having stored a computer program adapted to be executed by a processor that is to be installed in the information processing device. The presently disclosed subject matter also relates to a method of assisting assessment of a patient's status that is to be performed by at least one computing device.

In the clinical setting, scoring with multiple parameters is often performed in order to assess a patient's status. Specifically, a score is defined in accordance with a value and/or a condition of each parameter, so that the patient's status is determined by a total of the scores of the parameters. As an exemplary scoring system, an EWS (Early Warning Score) that is designed to detect deterioration of a patient's status at an early stage is known. U.S. Pat. No. 10,095,838 B2 discloses a device configured to calculate an EWS based on physiological signals acquired from a patient, and to visualize the same.

### SUMMARY

It is required to improve assistability for the assessment of the patient's status.

An illustrative aspect of the presently disclosed subject matter may provide an information processing device, comprising:
an interface configured to receive physiological information of a patient; and
a processor configured to:
   acquire contribution information corresponding to contributions of N first parameters (N is an integer of at least 2) with respect to a status of the patient based on the physiological information; and
   cause a visualizing device to visualize the contribution information under a condition that M first parameters (M is an integer of at least 2 and at most N) included in the N first parameters are substituted by L second parameter (L is an integer less than M).

An illustrative aspect of the presently disclosed subject matter may provide a server device adapted to communicate with a client device and comprising the information processing device described above, wherein the interface is configured to receive the physiological information from the client device.

An illustrative aspect of the presently disclosed subject matter may provide a non-transitory computer-readable medium having stored a computer program adapted to be executed by a processor installed in an information processing device, the computer program being configured to, when executed, cause the information processing device to:
receive physiological information of a patient;
acquire contribution information corresponding to contributions of N first parameters (N is an integer of at least 2) with respect to a status of the patient based on the physiological information; and
cause a visualizing device to visualize the contribution information under a condition that M first parameters (M is an integer of at least 2 and at most N) included in the N first parameters are substituted by L second parameter (L is an integer less than M).

An illustrative aspect of the presently disclosed subject matter may provide a method of assisting assessment of a patient's status executed by at least one computing device, the method comprising:
receiving physiological information of a patient;
acquiring contribution information corresponding to contributions of N first parameters (N is an integer of at least 2) with respect to a status of the patient based on the physiological information; and
causing a visualizing device to visualize the contribution information under a condition that M first parameters (M is an integer of at least 2 and at most N) included in the N first parameters are substituted by L second parameter (L is an integer less than M).

### DESCRIPTION OF THE DRAWINGS

FIG. 1 illustrates a configuration of a status assessment system according to an exemplary embodiment.

FIG. 2 illustrates a functional configuration of an information processing device adapted to be installed in a server device of FIG. 1.

FIG. 3 illustrates exemplary processing executed by a processor of FIG. 2.

FIG. 4 illustrates exemplary contribution information visualized by a visualizing device of FIG. 2.

FIG. 5 illustrates another exemplary contribution information visualized by the visualizing device of FIG. 2.

FIG. 6 is a diagram for explaining feature quantities related to electrocardiogram of FIG. 5.

FIG. 7 illustrates another exemplary contribution information visualized by the visualizing device of FIG. 2.

### DESCRIPTION OF EMBODIMENTS

Exemplary embodiments will be described in detail with reference to the accompanying drawings.

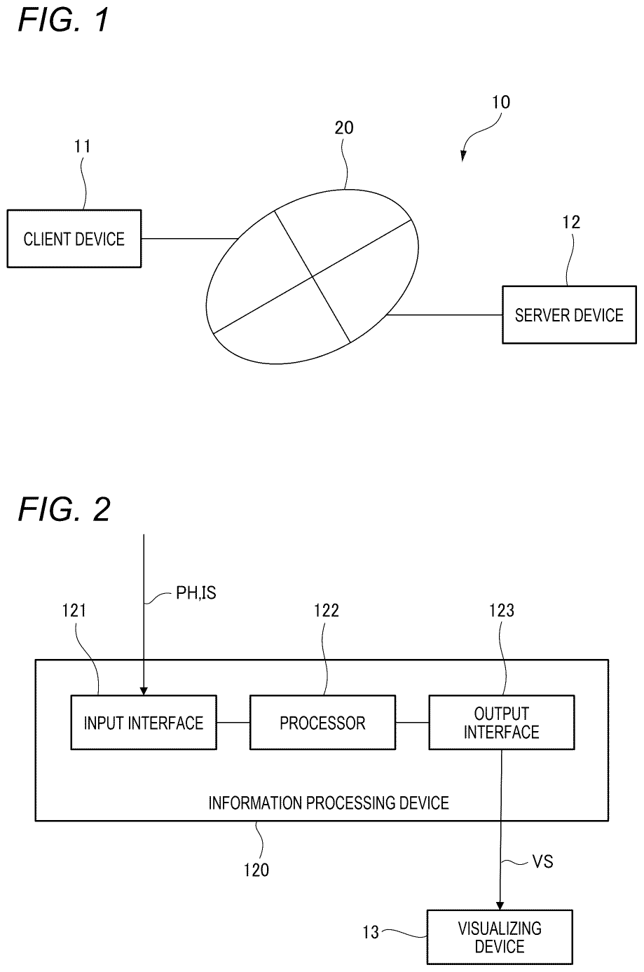

FIG. 1 illustrates a configuration of a status assessment system 10 according to an exemplary embodiment. The status assessment system 10 includes a client device 11 and a server device 12.

The client device 11 is a device configured to acquire physiological information of a patient. The expression “a device configured to acquire physiological information of a patient” as used herein is intended to encompass a device configured to directly receive signals corresponding to the physiological information from one or more sensors attached to the patient, as well as a device configured to finally receive data corresponding to the physiological information transmitted from such a device.

Accordingly, the number of client device 11 may be multiple. The same client device that is to be used by multiple users with different accounts may be regarded as multiple client devices. The client device 11 may be a stationary device that is installed in a specific place, or a portable device that can be carried by a user.

The server device 12 is installed in a place that is remote from the client device 11. The client device 11 and the server device 12 are configured to perform two-way communication of data via a communication network 20.

The client device 11 is configured to transmit physiological information data PH corresponding to the physiological information as acquired to the server device 12. The physiological information data PH may be provided in the form of analog data or digital data in accordance with the specification of the communication network 20.

As illustrated in FIG. 2, the server device 12 includes an information processing device 120. The information processing device 120 includes an input interface 121, a processor 122, and an output interface 123. The processor 122 is an exemplary computing device.

The input interface 121 is configured as a hardware interface that receives the physiological information data PH transmitted from the client device 11. In the case where the physiological information data PH is provided in the form of analog data, the input interface 121 is provided with an adequate conversion circuit including an A/D converter.

The processor 122 is configured to acquire, contribution information CT corresponding to contributions of multiple first parameters to the patient's status, based on the physiological information data PH.

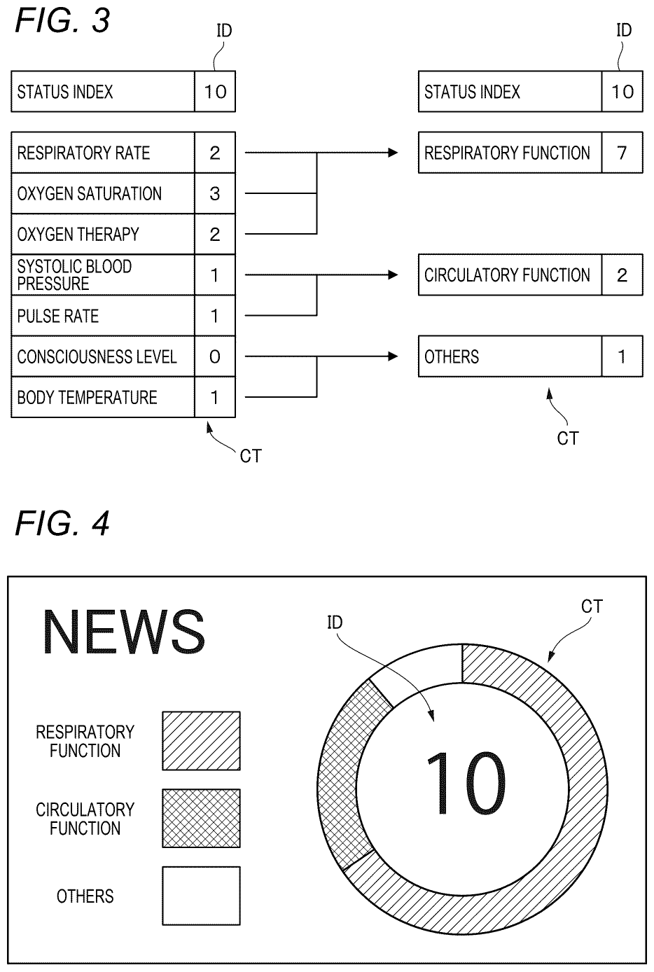

As an example, the processor 122 executes processing corresponding to an operation of inputting physiological information into a National Early Warning Score (NEWS) model. As illustrated in FIG. 3, the NEWS model is designed to represent states of multiple parameters that could contribute to the patient's status with scores, so that a total of the scores is acquired as an index ID representing the patient's status. A higher total score corresponds to a higher abnormality of the status. The higher score of each parameter in the contribution information CT corresponds to a higher contribution to the abnormality.

Accordingly, the physiological information data PH according to the present example is configured to include, as exemplary multiple first parameters, information indicating a respiratory rate, an oxygen saturation, an oxygen therapy (is performed or not), a systolic blood pressure, a pulse rate, a consciousness level, and a body temperature that are to be referred to in the NEWS score.

The processor 122 is configured to execute processing for substituting a second parameter for the first parameters at least partially. Namely, the number of the second parameter is less than the number of the first parameters.

In this example, three first parameters including the “respiratory rate”, the “oxygen saturation”, and the “oxygen therapy” are substituted by a second parameter named “respiratory function”. The score of the “respiratory function” coincides with a sum of the scores of the “respiratory rate”, the “oxygen saturation”, and the “oxygen therapy”.

Similarly, two first parameters including the “systolic blood pressure” and the “pulse rate” are substituted by a second parameter named “circulatory function”. The score of the “circulatory function” coincides with a sum of the scores of the “systolic blood pressure” and the “pulse rate”. In addition, the two first parameters including the “consciousness level” and the “body temperature” are substituted by a second parameter named “others”. The score of the “others” coincides with a sum of the scores of the “consciousness level” and the “body temperature”.

Namely, the amount corresponding to the total contribution is not changed before and after the parameter substitution. As a result, the value of the total score as an index indicating the patient's status is not changed as well.

In other words, at least M (M is an integer of at least 2 and at most N) first parameters included in N (N is an integer of at least 2) first parameters are substituted by one or more L (L is an integer less than M) second parameters. In this example, N=7, M=7, and L=3.

As illustrated in FIG. 2, the status assessment system 10 includes a visualizing device 13. The visualizing device 13 is configured to visualize an image including prescribed information. The visualizing device 13 may be realized by a display that displays the image, a projector that projects the image, a printer that prints the image, or the like. The visualizing device 13 may be a part of the client device 11, or may be a device independent of both the client device 11 and the server device 12.

The processor 122 is configured to output, from the output interface 123, visualizing data VS that causes the visualizing device 13 to visualize the contribution information CT under a condition that the first parameters are substituted at least partially with the second parameter. The visualizing data VS may be provided in the form of analog data or digital data according to the specification of the visualizing device 13.

The output interface 123 is configured as a hardware interface adapted to output the visualizing data VS. In the case where the visualizing data VS is provided in the form of analog data, the output interface 123 is provided with an adequate conversion circuit including a D/A converter.

FIG. 4 illustrates the contribution information CT that is visualized in the visualizing device 13. In this example, the total score “10” as the index ID indicating the patient's status is also subjected to the visualization. In this example, the contributions of the respective second parameters are visualized in the form of a pie chart.

An SOFA (Sequential Organic Failure Assessment) model can be exemplified as another model for acquiring a status index in which states of multiple parameters that could contribute to the patient's status are represented by scores to acquire an index representing the status as a total score thereof.

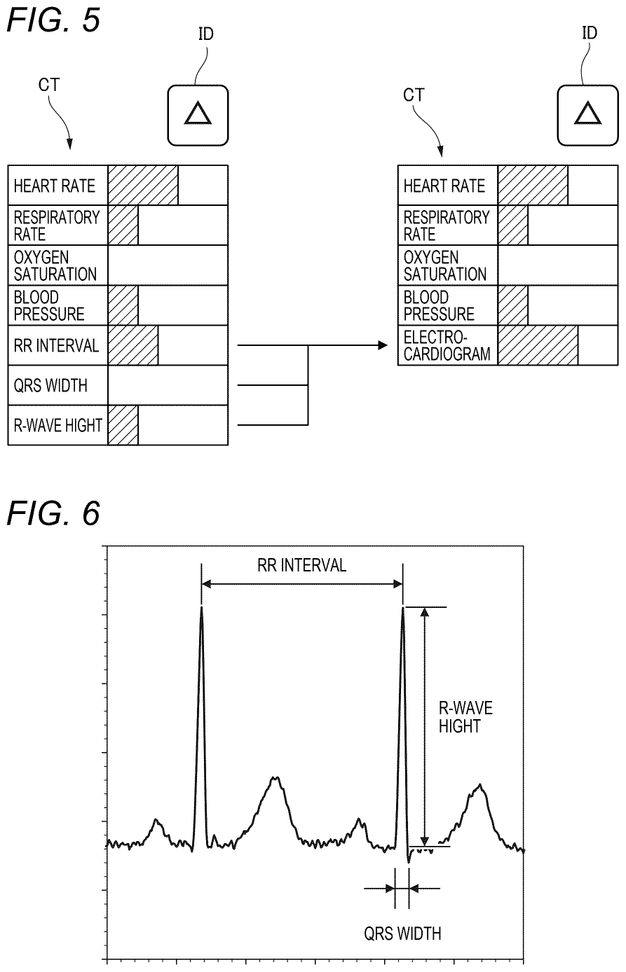

With reference to FIGS. 5 and 6, there will be described another exemplary processing for substituting a lesser number of second parameter for multiple first parameters at least partially.

In this example, the processor 122 of the information processing device 120 is configured to input the physiological information data PH into an explainable artificial intelligence (hereinafter, abbreviated as “explainable AI”) that is configured to estimate the patient's status.

As illustrated in FIG. 5, the explainable AI is configured to output, as an estimation result, an index ID indicating the patient's status. In addition, the explainable AI is configured to visualize contribution information CT indicating how much each of the multiple parameters has contributed to the estimation result.

The index ID according to this example is determined so as to represent the patient's status in three levels associated with figures including “circle”, “triangle”, and “cross”. The “circle” mark corresponds to a normal condition. The “triangle” mark as illustrated corresponds to a condition requiring attention. The “cross” mark corresponds to a condition requiring caution.

Accordingly, the physiological information data PH according to the present example is configured to include, as exemplary multiple first parameters, information indicating a heart rate, a respiratory rate, an oxygen saturation, a blood pressure, and multiple feature quantities in electrocardiogram that are adapted to be referred to or extracted by the explainable AI.

FIG. 6 illustrates exemplary feature quantities that are to be referred to or extracted. Specifically, an RR interval, a QRS width, and an R-wave height are referred to or extracted.

The explainable AI is implemented as an estimation model created by machine learning that is performed such that the model can output an index indicating a patient's status together with information indicating contribution of each of first parameters to the index in response to input of physiological information including the first parameters.

In this example as well, the processor 122 is configured to execute processing for substituting a second parameter for the first parameters at least partially. In other words, at least M (M is an integer of at least 2 and at most N) first parameters included in N (N is an integer of at least 2) first parameters are substituted by one or more L (L is an integer less than M) second parameters. In this example, N=7, M=3, and L=1.

Specifically, three first parameters including “RR interval”, “QRS width”, and “R-wave height” are substituted by a second parameter named “electrocardiogram”. The amount indicating the contribution of the “electrocardiogram” coincides with a sum of the amounts indicating the contributions of the “RR interval”, “QRS width”, and “R-wave height”. Namely, the amount corresponding to the total contribution is not changed before and after the parameter substitution. As a result, the index indicating the patient's status is not changed as well.

By acquiring information relating to the contribution of each of the first parameters to the patient's status, estimation of an influence thereof on the status change of the patient may be enabled. As the number of the first parameters is increased, a more detailed estimation as to which parameter has influenced the patient's status is enabled. However, immediate identification of the parameter related to the status change in question would be difficult. According to the configuration of the present exemplary embodiment, the first parameters included in the contribution information CT is at least partially substituted by a lesser number of second parameter, so that the number of parameters to be subjected to the observation can be reduced. Accordingly, even if the number of the first parameters is increased in order to enable a more detailed estimation of the change of the patient's status, it is possible to reduce the difficulty of identifying which parameter is related to the status change in question. As a result, it is possible to enhance the assistability for the assessment of the patient's status.

The processor 122 of the information processing device 120 according to the present exemplary embodiment changes the name of the second parameter that is subjected to the visualization by the visualizing device 13 to another name that encompasses the names of the M first parameters.

In the example described with reference to FIG. 3, the name of the second parameter “respiratory function” encompasses the names of three first parameters including the “respiratory rate,” the “oxygen saturation,” and the “oxygen therapy”. The substitution is based on a fact that each of the “respiratory rate,” the “oxygen saturation,” and the “oxygen therapy” is a parameter related to the “respiratory function”.

Similarly, the name of the second parameter “circulatory function” encompasses the names of the two first parameters including the “systolic blood pressure” and the “pulse rate”. The name of the second parameter “others” encompasses the names of the two first parameters including the “consciousness level” and the “body temperature”.

In the example described with reference to FIG. 5, the name of the second parameter “electrocardiogram” encompasses the names of three first parameters including the “RR interval,” the “QRS width,” and the “R-wave height”. This substitution is based on a fact that each of the “RR interval,” the “QRS width,” and the “R-wave height” is a parameter related to the feature quantity of the “electrocardiogram”.

According to the above configuration, it is possible to curb the expansion of an area where the name of the second parameter is visualized in an area for enabling the visualization of the contribution information, without impairing intuitive understanding with respect to the contributions of the first parameters before being subjected to the substitution to the second parameter.

Alternatively, the name of the second parameter may be determined as a name with a style listing the names of the first parameters prior to the substitution. For instance, the name of the second parameter “others” in the example described with reference to FIG. 3 may be determined as “consciousness level/body temperature”. According to the above configuration, it is possible to prompt the understanding of the first parameters that are subjected to the substitution with the second parameter.

The name of the second parameter with the style listing the names of the first parameters can be rephrased such that the name of the second parameter encompasses the names of the first parameters. Accordingly, the expression “another name that encompasses the names of the first parameters” as used herein is intended to mean a name of the second parameter that does not directly include the names of the first parameters based on assumption that a user understands the relationship between the first parameters and the second parameter.

Several policies may be conceived as to how to determine the first parameters to be subjected to the substitution with the second parameter.

As an example, substitution can be performed with respect to first parameters encompassed by a second parameter that can be involved in an event contributing to the change of the patient's status with a higher probability. For instance, a patient undergoing hospitalization tends to experience aspiration due to muscle weakness. This would involve pneumonia that often results in impairment of the “respiratory function”. In another case, a patient with postoperative or sepsis often experiences shock symptom caused by hypovolemia due to an inflammatory reaction. This would involve a comprehensive influence on the “circulatory function,” such as hypotension and an increase in the heart rate as a compensation.

Accordingly, in the example described with reference to FIG. 3, the first parameters including the “respiratory rate”, the “oxygen saturation”, and the “oxygen therapy”, which may be included in the “respiratory function” of the second parameter are preferentially selected to be substituted.

As the first parameters are substituted by the second parameter that can be involved an event contributing to the change of the patient's status with a higher probability, readability of the situation can be enhanced because the number of parameters that are subjected to the observation of the user can be reduced. In addition, since consideration as to which parameter has a higher contribution wound not be necessary with respect to such first parameters, it is possible to facilitate paying attention to the contributions of the remaining first parameters.

As another example, substitution can be performed with respect to first parameters encompassed by a second parameter that can be involved in an event contributing to the change of the patient's status with a lower probability.

In the example described with reference to FIG. 3, the second parameter having a lower probability that an event that contributes to the change of the patient's status is occurred is entitled as “others”, so that the first parameters including the “consciousness level” and the “body temperature” are preferentially selected to be substituted.

As the first parameters are substituted by the second parameter that can be involved an event contributing to the change of the patient's status with a higher probability, readability of the situation can be enhanced because the number of parameters that are subjected to the observation of the user can be reduced. In addition, since paying attention to such first parameters would not be necessary, it is possible to facilitate paying attention to parameters with higher contributions to the patient's status.

In the case of the example described with reference to FIG. 5, the “RR interval”, the “QRS width”, and the “R-wave height” that are not general items to be observed in normal clinical practice are preferentially selected as the first parameters to be substituted by the “electrocardiogram” that is an item to be observed as a so-called “vital sign”.

The first parameters to be substituted are determined by the processor 122 of the information processing device 120. The determination may be performed in advance, or may be dynamically performed in accordance with the contribution of each first parameter in the contribution information CT that is acquired in response to the input of the physiological information data PH. In either case, information defining a combination of multiple first parameters to be substituted and a single second parameter is stored in advance in a storage (not illustrated) so that the processor 122 can refer to the combination.

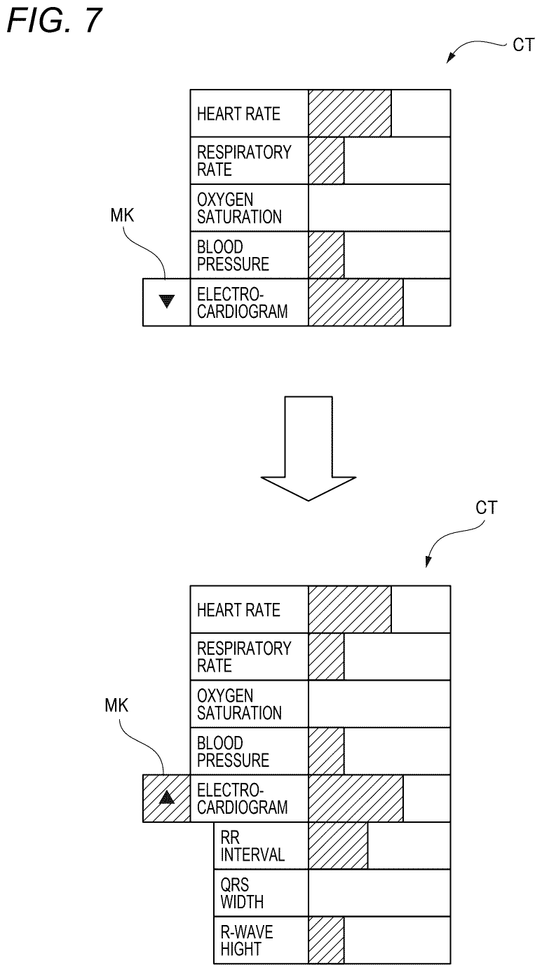

As illustrated in FIG. 7, the processor 122 of the information processing device 120 may visualize a marker MK indicating that the visualized parameter is the second parameter as a result of the substitution.

The way of visualization may be appropriately determined as long as it is possible to provide distinction with respect to first parameters that have not been subjected to the substitution. In addition to or in place of the marker MK, the color of characters, the size of characters, the font of characters, the background color, the background pattern, or the like may be appropriately changed. In the example illustrated in FIG. 4, distinguishable visualization is applied to the graph region representing contributions of the respective second parameters.

In this case, a user of the client device 11 can input, through an adequate user interface, an instruction for bringing an image of the marker MK into a selected state. As illustrated in FIG. 2, the input interface 121 of the information processing device 120 may receive instruction information IS corresponding to the instruction from the client device 11.

In accordance with the instruction information IS that is received by the input interface 121, the processor 122 may be configured to cause the visualizing device 13 to visualize, the first parameters prior to the substitution with the second parameter, in addition to the second parameter associated with the marker MK that is brought into the selected state.

In this example, the marker MK associated with the “electrocardiogram” as the second parameter is brought into the selected state. Accordingly, the “RR interval”, the “QRS width”, and the “R-wave height” as the first parameters prior to the substitution with the “electrocardiogram” are additionally visualized.

It should be noted that the first parameters prior to the substitution with the second parameter may be visualized in place of the second parameter associated with the marker MK brought into the selected state.

In response to the reception of the instruction information IS corresponding to an instruction to cancel the selected state of the marker MK by the input interface 121, the processor 122 returns the second parameter to the original visualization state.

According to such a configuration, since the information related to the contribution of the first parameters that is omitted to be visualized due to the substitution with the second parameter can be confirmed at any time, it is possible to balance the readability and reviewability of the contribution information.

The processor 122 of the information processing device 120 having various functions as exemplified above may be implemented by at least one versatile microprocessor configured to cooperate with at least one versatile memory. Examples of the versatile microprocessor include a CPU, an MPU, and a GPU. Examples of the versatile memory include a ROM, a RAM, and the like. In this case, a computer program that implements the various functions described above may be stored in the ROM. The ROM is an exemplary non-transitory computer-readable medium having stored a computer program. The versatile microprocessor designates at least a part of the program stored in the ROM, loads the designated program in the RAM, and executes the above-described processing in cooperation with the RAM. The computer program may be pre-installed in a versatile memory, or may be downloaded from an external server device with a communication network, and then installed in the versatile memory. In this case, the external server device is an exemplary non-transitory computer-readable medium having stored a computer program.

The processor 122 may be implemented by at least one exclusive integrated circuit capable of executing the computer program described above. Examples of the exclusive integrated circuit include a microcontroller, an ASIC, and an FPGA. In this case, the above-described computer program is pre-installed in the memory element included in the exclusive integrated circuit. The memory element is an exemplary non-transitory computer-readable medium having stored therein a computer program. The processor 122 may also be implemented by a combination of a versatile microprocessor and a exclusive integrated circuit.

The various configurations described above are merely illustrative for facilitating understanding of the presently disclosed subject matter. Each of the illustrative configurations may be appropriately modified or combined with another illustrative configuration within the scope of the present disclosure.

In the above exemplary embodiment, a specific first parameter is substituted by a single second parameter. However, as long as the amount corresponding to the total contribution is not changed before and after the substitution, a specific first parameter may be subjected to substitution with multiple second parameters. For example, the “oxygen saturation” as the first parameter illustrated in FIG. 3 may be related to both the “respiratory function” and the “circulatory function” as the second parameter.

In this case, a ratio in which the contribution of the “oxygen saturation” is substituted by the “respiratory function” and a ratio in which the contribution is substituted by the “circulatory function” may be separately defined. Alternatively, an overlap between the contribution substituted by the “respiratory function” and the contribution substituted by the “circulatory function” may be permitted. In the latter case, the visualization may be performed so as to make the overlapping portion apparent, for example, in the graph illustrated in FIG. 4.

In the above exemplary embodiment, as illustrated in FIGS. 4 and 5, the index ID indicating the patient's status is visualized together with the contribution information CT. However, the visualization of the index ID may be omitted.

As long as the contribution information CT corresponding to the contributions of the first parameters to the patient's status can be acquired, the physiological information data PH need not be inputted to the model involving the acquisition of the index ID indicating the patient's status.

In the above exemplary embodiment, the information processing device 120 is installed in the server device 12 that can communicate with the client device 11 over the communication network 20. However, the information processing device 120 may be installed in an adequate device that can acquire the physiological information PH. The information processing device 120 may be installed in the client device 11, or may be installed in a monitor device that directly receives a signal corresponding to the physiological information from a sensor attached to the patient.

The processing for acquiring the contribution information CT, the processing for substituting a single second parameter for multiple first parameters, and the processing for causing the visualizing device 13 to visualize the second parameter are not necessarily executed by the same device. The multiple kinds of processing may be shared by the client device 11 and the server device 12.

1. An information processing device, comprising:
an interface configured to receive physiological information of a patient; and
a processor configured to:
acquire contribution information corresponding to contributions of N first parameters (N is an integer of at least 2) with respect to a status of the patient based on the physiological information; and
cause a visualizing device to visualize the contribution information under a condition that M first parameters (M is an integer of at least 2 and at most N) included in the N first parameters are substituted by L second parameter (L is an integer less than M).

2. The information processing device according to claim 1,
wherein the processor is configured to cause the visualizing device to visualize name of the second parameter as another name for encompassing names of the M first parameters.

3. The information processing device according to claim 1,
wherein the physiological information is inputted into a model for acquiring an index indicating the status of the patient in accordance with respective contributions of the N first parameters.

4. The information processing device according to claim 3,
wherein the model is an explainable artificial intelligence that is created by machine learning so as to output the contribution information in response to input of the physiological information.

5. The information processing device according to claim 4,
wherein the M first parameters are associated with feature quantities that are extracted from the physiological information by the explainable artificial intelligence to perform estimation.

6. The information processing device according to claim 3,
wherein the processor is configured to cause the visualizing device to visualize the index together with the L second parameter.

7. The information processing device according to claim 1,
wherein the processor is configured to determine the M first parameters as first parameters encompassed by the second parameter that can be involved in an event contributing to a status change of the patient with a higher probability.

8. The information processing device according to claim 1,
wherein the processor is configured to determine the M first parameters as first parameters encompassed by the second parameter that can be involved in an event contributing to a status change of the patient with a lower probability.

9. The information processing device according to claim 1,
wherein the processor is configured to cause the visualizing device to visualize the M first parameters in addition to or in place of the L second parameter, in response to reception of information corresponding to a user's instruction by the interface.

10. A server device adapted to communicate with a client device and comprising t information processing device according to claim 1,
wherein the interface is configured to receive the physiological information from the client device.

11. A non-transitory computer-readable medium having stored a computer program adapted to be executed by a processor installed in an information processing device, the computer program being configured to, when executed, cause the information processing device to:
receive physiological information of a patient;
acquire contribution information corresponding to contributions of N first parameters (N is an integer of at least 2) with respect to a status of the patient based on the physiological information; and
cause a visualizing device to visualize the contribution information under a condition that M first parameters (M is an integer of at least 2 and at most N) included in the N first parameters are substituted by L second parameter (L is an integer less than M).

12. A method of assisting assessment of a patient's status executed by at least one computing device, the method comprising:
receiving physiological information of a patient;
acquiring contribution information corresponding to contributions of N first parameters (N is an integer of at least 2) with respect to a status of the patient based on the physiological information; and
causing a visualizing device to visualize the contribution information under a condition that M first parameters (M is an integer of at least 2 and at most N) included in the N first parameters are substituted by L second parameter (L is an integer less than M).
